(19) United States
(12) Patent Application Publication
(10) Pub. No.: US 2025/0255588 A1
(43) Pub. Date: Aug. 14, 2025

(54) URINE SAMPLE COLLECTION DEVICE

(71) Applicant: FloGO Products Ltd, Purley Surrey (GB)

(72) Inventor: Lisa MALYON, Purley Surrey (GB)

(21) Appl. No.: 18/437,045

(22) Filed: Feb. 8, 2024

(51) Int. Cl. A61B 10/00; A61F 5/44; A61F 5/451

(52) U.S. Cl. CPC A61B 10/007; A61F 5/4408; A61F 5/451

(57) ABSTRACT

There is a provided a urine collection device for collecting an uncontaminated urine sample from a pre-continent or an incontinent user, the urine collection device comprising: a receptacle configured to be placed within a nappy or an incontinence pad of the user to receive and retain a urine sample collected from the user in use, wherein the receptacle comprises: a receiving portion for receiving the urine sample, the receiving portion comprising an opening configured to engage with a human body portion surrounding external genitalia of the user, thereby enabling urine exiting the body of the user to be captured directly in use; a reservoir for retaining the received urine sample in use; and an outlet for draining the retained urine sample in use, wherein the opening is larger than the outlet.

### TECHNICAL FIELD

The present invention relates to a urine sample collection device for collecting an uncontaminated urine sample from a pre-continent or an incontinent user.

### BACKGROUND

Obtaining an uncontaminated urine sample quickly can be the difference between life and death. Urine sample collection is important for health-related testing purposes, such as to test for urinary tract infections (UTIs), to diagnose diabetes, or to diagnose more life-threatening blood diseases such as sepsis. After diagnosis and treatment, more urine samples are required to monitor the patient's health and response to treatment.

Uncontaminated urine samples are particularly challenging to collect from pre-continent patients, e.g., nappy-wearing infants, and incontinent patients, e.g., elderly or disabled people wearing incontinence pads or incontinence pants, as these patients cannot sit over a toilet to collect a sample, either through lack of mobility, or being unable to urinate on demand.

The only non-invasive solutions for collecting a urine sample from pre-continent and/or incontinent patients are: 1) the urine collection bag; 2) the ‘Newcastle Pad’; and 3) the ‘Clean Catch’.

A urine collection bag, which is usually for use with infants, is a small plastic bag, typically made from polyethylene, with an adhesive section around the opening which fits over the user's genital area. The adhesive section is attached to the patient and then their pre-continent or incontinent wear is secured in place. In order to collect urine for testing purposes after the patient has urinated, the bag has to be removed carefully from the patient's genitals and then the urine has to be emptied from the bag into a urine specimen container via the bag opening. The main problems with the urine collection bag are that there is very little space between the genitals and the bag, and the urine sample comes into contact with the patient's genitals during collection. This leads to the urine sample being contaminated, for example with skin flora and/or faeces. The urine collection bag therefore does not enable an uncontaminated urine sample to be collected from the patient.

A ‘Newcastle Pad’ is a sponge or pad that is placed into the incontinence wear of the patient. Once a urine sample has been absorbed into the Newcastle Pad, the Newcastle Pad is removed from the incontinence wear and the urine is then extracted using a syringe and transferred into a urine specimen container for testing. The main problem with the Newcastle Pad is contamination of the urine sample with skin flora since the pad is in direct contact with the patient's body. Another problem with the Newcastle Pad is that the process of collecting the urine sample is time-consuming, messy and often needs repeating.

The ‘Clean Catch’ is a bowl that is held underneath the undressed patient in order to catch a urine sample. A carer or healthcare provider has to wait until the undressed patient urinates and then attempt to catch enough of the urine to form the sample. There are several problems with using such a device, namely the process is extremely messy, time-consuming, impractical and takes up valuable bed space in a healthcare environment.

All three of the above-described devices are therefore highly ineffective. The urine collection bag and the Newcastle Pad in particular both result in high levels of contamination, e.g., by skin flora and/or faeces, of the urine sample and are therefore regarded as ineffective solutions for collecting an uncontaminated urine sample from pre-continent or incontinent users. The difficulties in obtaining an uncontaminated urine sample from pre-continent or incontinent users can also result in unnecessary antibiotic treatment because of false test results due to contaminated urine samples, which leads to greater antimicrobial resistance.

As a result of these issues with the above devices, intrusive catheterisation often becomes the default method to obtain an uncontaminated urine sample from pre-continent or incontinent users. This involves inserting a catheter into the patient's bladder to collect the urine. This is a highly invasive procedure which can be painful and uncomfortable. It can also increase the chance of infection in patients and lead to longer hospital stays.

The present invention has been devised to mitigate or overcome the above-mentioned problems.

### SUMMARY OF THE INVENTION

According to the present invention, there is provided a urine collection device for collecting an uncontaminated urine sample from a pre-continent or an incontinent user, the urine collection device comprising: a receptacle configured to be placed within a nappy or an incontinence pad of the user to receive and retain a urine sample collected from the user in use, wherein the receptacle comprises: a receiving portion for receiving the urine sample, the receiving portion comprising an opening configured to engage with a human body portion surrounding external genitalia of the user, thereby enabling urine exiting the body of the user to be captured directly in use; a reservoir for retaining the received urine sample in use; and an outlet for draining the retained urine sample in use, wherein the opening is larger than the outlet.

The urine collection device enables an uncontaminated urine sample to be collected. The urine collection device is suitable for collecting uncontaminated urine samples from pre-continent patients, e.g., nappy-wearing infants, and incontinent patients, e.g., elderly or disabled people wearing incontinence wear such as incontinence pads or incontinence pants. The urine collection device is particularly suitable for collecting uncontaminated urine samples from pre-continent males and females, and incontinent female users.

The receptacle is configured to be placed within a nappy or an incontinence pad of the user, such that use of the device is both discrete and comfortable. It should be noted that the receptacle is configured to be placed within any other object that is absorbent enough to capture excess urine, e.g., a sanitary pad, sanitary pants, or incontinence pants, etc. The opening of the receiving portion is configured to engage with a human body portion surrounding external genitalia of the user, such that urine exiting the body of the user can be captured directly. This prevents contact of the urine sample with the user's genitalia or skin during collection. The reservoir enables the received urine sample to be retained within the receptacle, which prevents contact of the urine sample with the user's genitalia after collection. Prevention of contact of the urine sample with the user's genitalia during and after collection prevents the urine sample from being contaminated with flora of the user's body, bacteria and/or faeces. The outlet enables the retained urine sample to be drained from the receptacle whilst maintaining an uncontaminated urine sample because the urine sample does not need to travel via the opening to be drained from the receptacle. The opening is larger than the outlet, which enables urine exiting the body of the user to be captured directly whilst also enabling the urine sample that is retained in the reservoir to be drained and collected for testing.

Advantageously, the present invention thereby enables the collection of an uncontaminated urine sample quickly, easily and discreetly. A further advantage of the present invention is that it enables an uncontaminated urine sample to be collected in a non-invasive manner. Efficient collection of uncontaminated urine samples as provided by the present invention provides significant benefits in improving testing and monitoring of health concerns such as UTIs, as it not only leads to more reliable results but also reduces the amount of time and resources wasted in repeating sample collection.

The receptacle may be between approximately 10 cm and approximately 17 cm in length, between approximately 4 cm and approximately 10 cm in width, and between approximately 3 cm and approximately 8 cm in height.

The receptacle may be between approximately 10 cm and approximately 12 cm in length, between approximately 4 cm and approximately 6 cm in width, and between approximately 3 cm and approximately 6 cm in height. These dimensions enable the urine collection device to be particularly suitable for collecting uncontaminated urine samples from pre-continent users, namely babies and infants.

The receptacle may be between approximately 15 cm and approximately 17 cm in length, between approximately 5 cm and approximately 10 cm in width, and between approximately 4 cm and 8 cm in height. More preferably, the receptacle may be between approximately 5 cm and approximately 7 cm in width. These dimensions enable the urine collection device to be particularly suitable for collecting uncontaminated urine samples from incontinent users, namely adults.

The receptacle may have a greater height dimension at a forward-facing end than a rearward-facing end, wherein the outlet may be positioned at the rearward-facing end. Advantageously, this provides an ergonomically-shaped opening and a better fit of the opening around the user's genitalia. This feature also enables urine exiting the body to be captured within the receptacle even whilst the user is moving or sitting to one side.

The receiving portion may comprise a rim configured to engage with the human body portion surrounding external genitalia of the user. Advantageously, the rim provides further comfort for the user when the urine collection device is in use.

The reservoir may comprise a substantially flat base. Advantageously, the substantially flat base enables the receptacle to stand on a surface, either before or after urine sample collection. In addition, the substantially flat base enables multiple receptacles to be stacked which provides space-saving and storage benefits.

The outlet may be in alignment with the substantially flat base. This provides further comfort for the user when the urine collection device is placed within the nappy or incontinence pad.

The outlet may include a temporary seal. Advantageously, the temporary seal prevents the urine sample from escaping the receptacle when the receptacle is inside the nappy or incontinence pad. When the receptacle is removed from the nappy or incontinence pad, the temporary seal can be broken in order to drain and collect the uncontaminated urine sample for testing.

The receptacle may comprise at least one overflow opening for releasing excess urine in use. Advantageously, during collection of a urine sample, the at least one overflow opening enables excess urine to be released from the receptacle once the urine sample reaches the level of the at least one overflow opening. This prevents the urine sample from reaching a level at which contact is made with the user, and thereby provides additional protection against contamination of the urine sample. The at least one overflow opening also ensures that the level of the urine sample does not exceed a maximum fill line of the reservoir.

The urine collection device may further comprise a mesh guard at the receiving portion for separating the urine collected in the reservoir from the external genitalia of the user in use. Advantageously, the mesh guard enables the urine collected in the reservoir to be separated from the external genitalia of the user in use. Using a mesh guard as part of the urine collection device enables the urine collection device to be particularly suitable for collecting uncontaminated urine samples from larger pre-continent male users and incontinent male users since the mesh guard acts as a barrier to prevent male genitalia from entering the reservoir.

The at least one overflow opening may be located between the mesh guard and the reservoir. Advantageously, this enables excess urine to be released via the at least one overflow opening before the level of the urine sample reaches the mesh guard, which further prevents contact of the urine sample with the user's genitalia or skin.

The mesh guard may be attached to the receptacle using a press fit fitting. Advantageously, this enables quick assembly and disassembly of the mesh guard with the receptacle. In addition, this attachment mechanism has no moving parts and is easily provided by features of the receptacle walls that may be formed by way of injection moulding.

The receptacle may comprise one or more engagement clips and the mesh guard may comprise one or more support clips for attaching the mesh guard to the receptacle. This attachment mechanism provides a sturdy attachment of the mesh guard to the receptacle.

The urine collection device may further comprise a tube, a first end of the tube being connected to the outlet, for directing the retained urine sample away from the reservoir in use.

The receptacle, the mesh guard and/or the tube may comprise a flexible material.

The flexible material of the receptacle may have a variable degree of flexibility characteristic and the degree of flexibility of the receptacle at the receiving portion may be greater than the degree of flexibility of the receptacle at the reservoir. Advantageously, the greater flexibility at the receiving portion provides further comfort for the user, and the greater thickness at the reservoir provides a more stable connection between the outlet and the tube. The receptacle may be flexible yet sturdy enough to be placed comfortably within undergarments such as nappies, incontinence pads or incontinence pants without collapsing onto the genitalia or skin.

The receptacle, the mesh guard and/or the tube may comprise medical-grade silicone. Advantageously, medical-grade silicone is skin-contact safe, durable, flexible, and soft to the touch.

The urine collection device may further comprise a collection container, a second end of the tube being connected to the collection container, for collecting the urine sample in use.

The first end of the tube may be sealed to the outlet and the second end of the tube may be sealed to the collection container. Advantageously, this prevents leakage of the urine sample from the urine collection device.

The collection container may comprise an inlet valve at an inlet end of the collection container.

The inlet valve may be a non-return valve. Advantageously, the non-return inlet valve at the inlet end of the collection container prevents urine from being transported back into the tube and back to the receptacle.

The collection container may further comprise an outlet valve at an outlet end of the collection container.

The outlet valve may be a non-return valve. Advantageously, the non-return outlet valve at the outlet end of the collection container prevents urine from being transported back into the collection container.

The urine collection device may further comprise a stop tap for controlling flow of the collected urine sample out of the collection container in use. Advantageously, this enables the urine sample to be removed from the collection container easily and hygienically.

The stop tap may be sealed to the collection container. Advantageously, this further prevents leakage of the urine sample from the urine collection device.

The collection container may be a flexible pouch. Advantageously, a flexible pouch collection container is lightweight and easy to transport.

The flexible pouch may comprise sealed edges. Advantageously, this further prevents leakage of the urine sample from the urine collection device.

The collection container may comprise an object support structure for mounting the collection container to a nearby object in use.

The object support structure may comprise a flat-fold hook. In an open configuration, the flat-fold hook enables the collection container to be mounted onto an object, such as a bed frame or to the back of a nappy, when the urine collection device is in use in order to keep the collection container stable and/or out of sight. In a closed configuration, flat-fold hook can be folded into alignment such that the urine collection device can be stored more efficiently.

The object support structure may be situated at the first end of the collection container.

The collection container may comprise a user support structure for mounting the collection container to an item of clothing of the user in use.

The user support structure may comprise a plurality of support slots.

The plurality of support slots may comprise a first support slot at a first side of the collection container and a second support slot at a second side of the collection container, wherein the first support slot and the second support slot are configured to receive a strap for mounting the collection container to the user in use. For example, the strap may be for mounting the collection container to a leg or the waist of the user.

The collection container may be transparent. Advantageously, the transparency enables the amount of urine that has been collected to be viewed and monitored.

The collection container may comprise polyvinyl chloride (PVC). Advantageously, PVC provides a suitable material that is tough, durable and economical.

The urine collection device may be configured to be disposable. This helps to ensure that a clean and uncontaminated urine collection device is used each time a urine sample is being collected.

Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

Specific embodiments of the present invention will now be described, by way of example only, with reference to the accompanying drawings, in which:

FIG. 1 is a perspective view of a urine collection device, in accordance with a first embodiment of the present invention;

FIG. 2 is an exploded view of the urine collection device shown in FIG. 1;

FIG. 3 is a front-left angled perspective view of the receptacle of the urine collection device shown in FIG. 1;

FIG. 4 is a front-right angled perspective view of the receptacle shown in FIG. 1;

FIG. 5 is rear-right perspective view of the receptacle shown in FIG. 1;

FIG. 6 is a top view of the receptacle shown in FIG. 1;

FIG. 7 is a side view of the receptacle shown in FIG. 1;

FIG. 8 is a rear view of the receptacle shown in FIG. 1;

FIG. 9 is a front view of a collection container shown in FIG. 1;

FIG. 10 is a side view of the collection container shown in FIG. 1;

FIG. 11 is an exploded view of a urine collection device, in accordance with a second embodiment of the present invention;

FIG. 12 is a perspective view of the receptacle shown in FIG. 11, wherein the receptacle is fitted with a mesh guard; and

FIG. 13 is an exploded view of the receptacle shown in FIG. 11, wherein the receptacle is fitted with a mesh guard.

### DETAILED DESCRIPTION

The present invention provides a urine collection device for collecting an uncontaminated urine sample from a pre-continent or an incontinent user, the urine collection device comprising: a receptacle configured to be placed within a nappy or an incontinence pad of the user to receive and retain a urine sample collected from the user in use, wherein the receptacle comprises: a receiving portion for receiving the urine sample, the receiving portion comprising an opening configured to engage with a human body portion surrounding external genitalia of the user, thereby enabling urine exiting the body of the user to be captured directly in use; a reservoir for retaining the received urine sample in use; and an outlet for draining the retained urine sample in use, wherein the opening is larger than the outlet.

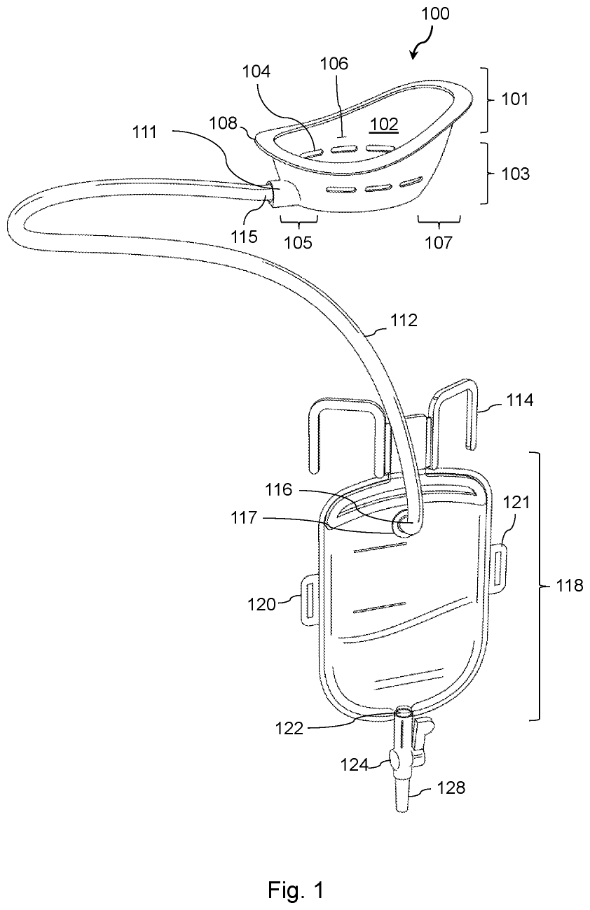

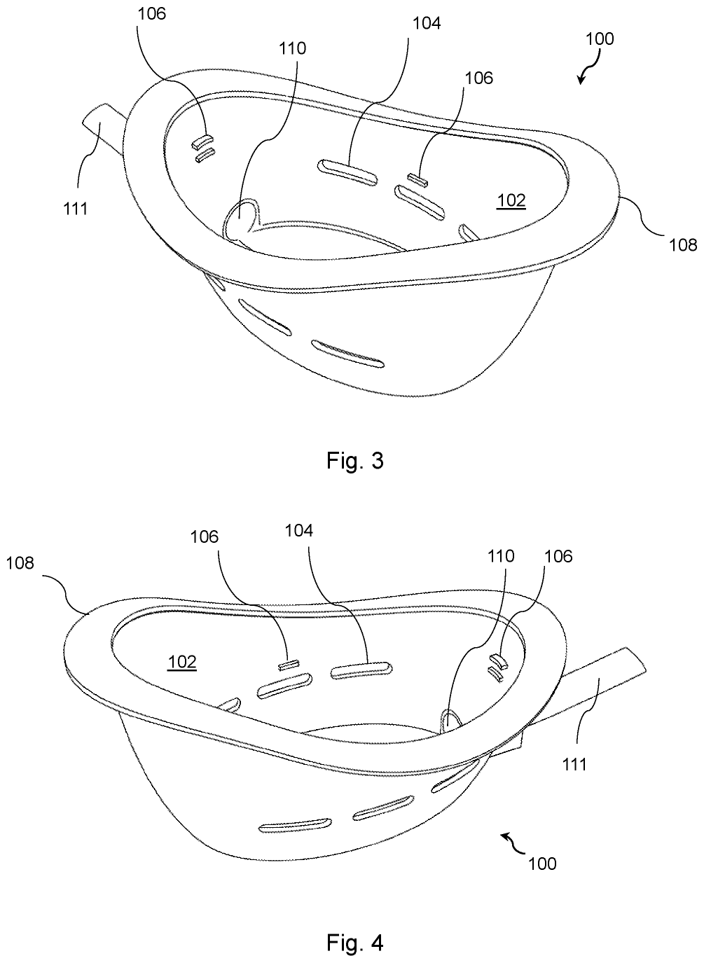

Specific embodiments of the present invention will now be described with reference to FIGS. 1 to 13.

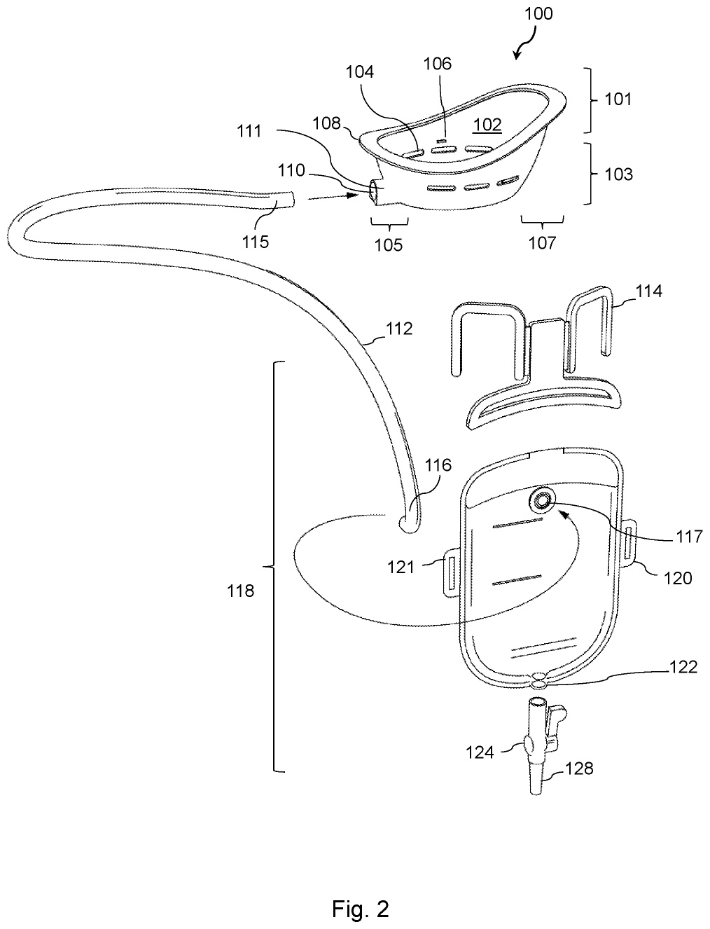

FIG. 1 shows a urine collection device, in accordance with a first embodiment of the present invention. The urine collection device comprises a receptacle 100 configured to be placed within a nappy or an incontinence pad of its user to receive and retain a urine sample. The receptacle 100 comprises a receiving portion 101 for receiving the urine sample. The receiving portion 101 comprises an opening 102 configured to engage with a human body portion surrounding external genitalia of the user (not shown). The opening 102 enables urine exiting the body of the user to be captured directly in use. The receptacle 100 also comprises a reservoir 103 for retaining the received urine sample in use. The receptacle 100 also comprises an outlet 110, which can be seen in FIG. 2, for draining the retained urine sample in use, wherein the opening 102 is larger than the outlet 110.

An exploded view of the urine collection device is shown in FIG. 2. The exploded view illustrates each component of the urine collection device before being sealed into place. When each component is sealed into place, the urine collection device provides a pre-scaled and packaged solution for collecting an uncontaminated urine sample for health professionals, carers, or parents of pre-continent and/or incontinent users to use.

The urine collection device enables an uncontaminated urine sample to be collected. The urine collection device is suitable for collecting uncontaminated urine samples from pre-continent patients, e.g., nappy-wearing infants, and incontinent patients, e.g., elderly or disabled people wearing incontinence pads or incontinence pants. The urine collection device is particularly suitable for collecting uncontaminated urine samples from pre-continent male and female users, and incontinent female users.

In use, the receptacle 100 is placed within a nappy or an incontinence pad of a user. The opening 102 of the receiving portion 101 is configured to engage with a human body portion surrounding external genitalia of the user. Urine exiting the body of the user is captured directly by the receptacle 100, without contacting the user's genitalia or skin during collection. The urine sample is received and retained by the reservoir 103. This prevents contact of the urine sample with the user's genitalia or skin after collection. The receptacle 100 can then be removed from the nappy or incontinence pad and the outlet 110 can be used to drain and collect the uncontaminated urine sample for testing.

Further advantageous and optional features of the urine collection device will now be described with reference to the figures.

The receptacle 100 shown in FIG. 1 has a greater height dimension at a forward-facing end 107 than a rearward-facing end 105, where the outlet 110 is positioned at the rearward-facing end 105. This provides an ergonomically-shaped opening 102 and a better fit of the opening 102 around the user's genitalia and enables urine exiting the body to be captured within the receptacle 100 even whilst the user is moving or sitting to one side. This feature can also be seen in the views provided in FIGS. 2 to 8. The receptacle 100 shown in FIGS. 1 to 8 has a height at the forward-facing end 107 of approximately 5.6 cm and a height at the rearward-facing end 105 of approximately 3.6 cm. The receptacle 100 has a length of approximately 11.8 cm, and a width of approximately 4.5 cm at the reservoir 103 of the receptacle 100 and a width of approximately 7.2 cm at the receiving portion 101 of the receptacle 100. FIG. 6 emphasises the overall substantially oval shape of the receptacle. In embodiments, other suitable shapes may be used. The receptacle 100 is comfortable and ergonomically-shaped. The shape of the receptacle 100 also ensures that the external genitalia of the user are surrounded, and that the anus of the user is avoided so as to prevent the urine being contaminated with faeces, body flora or skin contaminants.

The dimensions of the receptacle 100 may vary based on the intended use of the urine collection device. The receptacle 100 may be between approximately 10 cm and approximately 17 cm in length, between approximately 4 cm and approximately 10 cm in width, and between approximately 3 cm and approximately 8 cm in height.

For pre-continent users, the receptacle may be preferably between approximately 10 cm and approximately 12 cm in length, between approximately 4 cm and approximately 6 cm in width, and between approximately 3 cm and approximately 6 cm in height. These dimensions enable the urine collection device to be particularly suitable for collecting uncontaminated urine samples from pre-continent users, namely babies and infants.

For incontinent users, the receptacle may be preferably between approximately 15 cm and approximately 17 cm in length, between approximately 5 cm and approximately 10 cm in width, and between approximately 4 cm and 8 cm in height. More preferably, the receptacle may be between approximately 5 cm and approximately 7 cm in width. These dimensions enable the urine collection device to be particularly suitable for collecting uncontaminated urine samples from incontinent users, namely adults.

It should be noted that the size and shape of the receptacle is by no means limited to the size and shape of the receptacle as described herein with reference to FIGS. 1 to 13. For example, the receptacle 100 may be substantially circular rather than substantially oval in shape. The receptacle 100 may be sized differently depending on the intended use of the urine sample collection device. The receptacle 100 may be formed using injection moulding techniques.

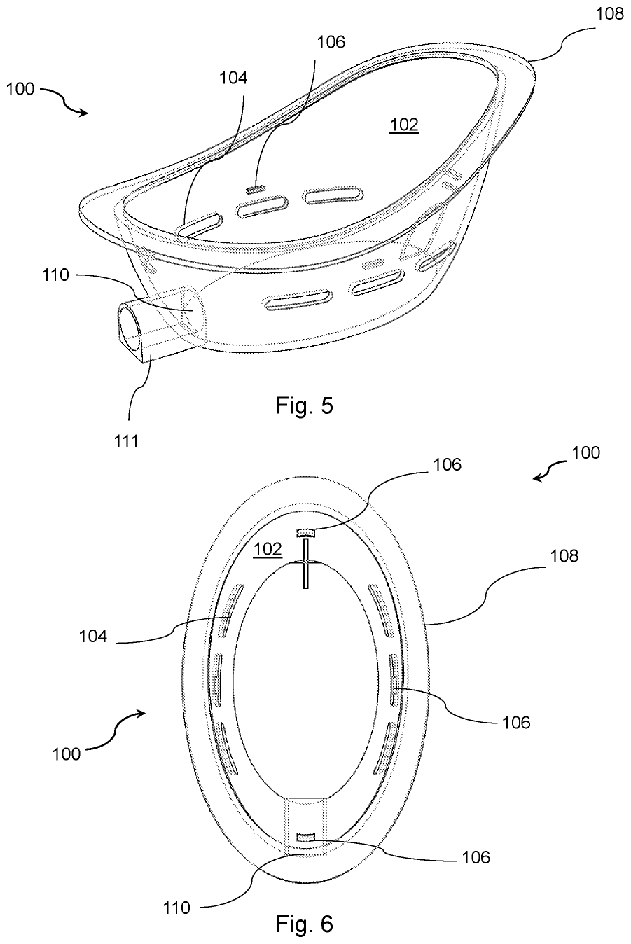

The receiving portion 101 includes a rim 108 configured to engage with the human body portion surrounding external genitalia of the user. The rim 108 may be a flat rim which protrudes from the opening 102. FIG. 5 in particular shows the opening 102 and the rim 108, the rim 108 being sloped and protruding from the opening 102. In use, the anterior of the rim 108 meets the pubic bone and the posterior of the rim 108 surrounds the back of the user's genitalia. The rim 108 provides further comfort for the user when the urine collection device is in use. In embodiments, the rim at the receiving portion may take an alternative form, e.g., a raised rim or a cushioned rim.

The reservoir 103 comprises a substantially flat base. The substantially flat base enables the receptacle to stand on a surface, either before or after urine sample collection. In addition, the substantially flat base enables multiple receptacles to be stacked which provides space-saving and storage benefits. The outlet 110 is in alignment with the substantially flat base.

The outlet 110 is substantially circular in shape. In embodiments, the outlet 110 may form other shapes, e.g., square, oval etc. The outlet 110 may include a temporary seal to prevent the urine sample from escaping the receptacle 100 when the receptacle 100 is inside the nappy or incontinence pad. When the receptacle 100 is removed from the nappy or incontinence pad, the temporary seal can be broken in order to drain and collect the uncontaminated urine sample for testing.

The receptacle 100 includes at least one overflow opening 104 for releasing excess urine in use. The at least one overflow opening 104 may be in the form of at least one overflow slot. The receptacle 100 shown in FIGS. 1 to 8 includes a total of six overflow slots 104. In embodiments, fewer or more overflow slots may be used. The overflow slots 104 are provided on the walls of the reservoir 103, with three overflow slots 104 residing on one side of the reservoir 103 and three overflow slots 104 residing on the other side of the reservoir 103. The overflow slots 104 shown in FIGS. 1 to 8 each have a width of approximately 1.5 cm and a height of approximately 0.3 cm. The dimensions and shape of the overflow slots may vary. During collection of a urine sample, the overflow slots 104 enable excess urine to be released from the receptacle once the urine sample reaches the level of the overflow slots 104. This prevents the urine sample from reaching a level at which contact is made with the user, and thereby provides additional protection against contamination of the urine sample. The overflow slots 104 also ensure that the level of the urine sample does not exceed a maximum fill line of the reservoir 103. Urine that reaches the overflow slots 104 will exit through the overflow slots 104 and into the user's nappy or incontinence pad/pants. Different configurations of overflow slots may be used. For example, the overflow slots may be arranged with two overflow slots on each side of the reservoir.

The receptacle 100 includes one or more engagement clips 106 for attaching a mesh guard to the receptacle 100. The receptacle 100 shown in FIGS. 1 to 8 includes a total of four engagement clips 106. In embodiments, fewer or more engagement clips may be used. Different configurations of engagement clips may be used.

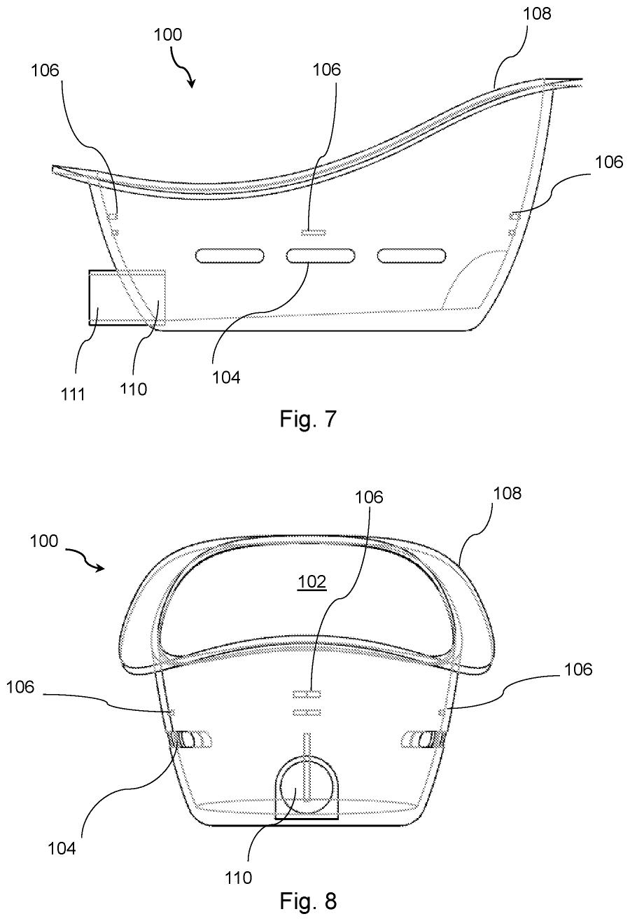

The urine collection device shown in FIGS. 1 to 8 includes a tube 112. A first end 115 of the tube 112 is connected to the outlet 110 of the receptacle 100. The tube 112 can be attached to the receptacle 100 by push fitting the tube 112 into the outlet 110. The tube 112 enables the retained urine sample to be directed away from the reservoir 103 in use. The thickness of the receptacle wall may be greater at the reservoir 103 of the receptacle 100 in comparison to the thickness of the receptacle wall at the receiving portion 101. The receptacle 100 comprises a flexible material. The flexible material of the receptacle 100 has a variable degree of flexibility characteristic and the degree of flexibility at the receiving portion 101 is therefore greater than the degree of flexibility of the receptacle 100 at the reservoir 103. The greater flexibility at the receiving portion 101 provides further comfort for the user, and the greater thickness at the reservoir 103 provides a more stable connection between the outlet 110 and the tube 112. The receptacle 100 is flexible yet sturdy enough to be placed comfortably within undergarments such as nappies, incontinence pads or incontinence pants without collapsing onto the genitalia or skin. As shown in FIG. 7, the interior of the base of the receptacle 100 may be sloped with respect to the exterior of the base of the receptacle 100. This aids the flow of the urine sample towards the outlet 110 and into the tube 112 and prevents urine from becoming stagnant within the reservoir 103. The receptacle 100 may include a non-return valve at the outlet 110 to prevent urine from travelling from the tube 112 back into receptacle 100.

The tube 112 may be suitably sized and shaped to enable the flow of urine outwardly from the receptacle 100. For example, the tube may have an outer diameter of approximately 9.6 mm and an inner diameter of approximately 6.4 mm. The tube may have an outer diameter between approximately 9.2 mm and 9.8 mm and an inner diameter between approximately 6.2 mm and approximately 6.8 mm. The tube may have a length of approximately 6 cm. The length of the tube may vary depending on the intended use of the urine collection device. For example, the length of the tube may be between approximately 2 cm and approximately 10 cm. The receptacle 100 may comprise a connector portion 111 in alignment with the outlet 110. The connector portion 111 enables the tube 112 to be connected to the receptacle 100 more easily. The size and shape of the connector portion 111 may vary based on the size and shape of the tube 112 being used. The connector portion 111 may form part of the moulded design of the receptacle 100. Alternatively, the connector portion 111 may be formed separately and attached to the receptacle 100. The tube 112 can be attached to the receptacle 100 by push fitting the tube 112 into the connector portion 111. The outlet 110 and/or the connector portion 111 may be sized and shaped suitably such that the tube 112 can be fitted into or over the outlet 110 and/or the connector portion 111.

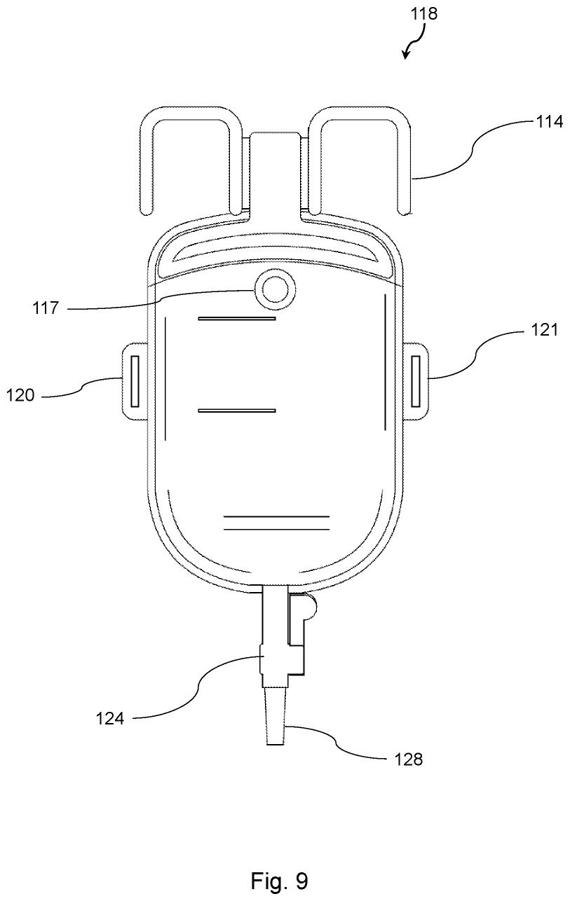

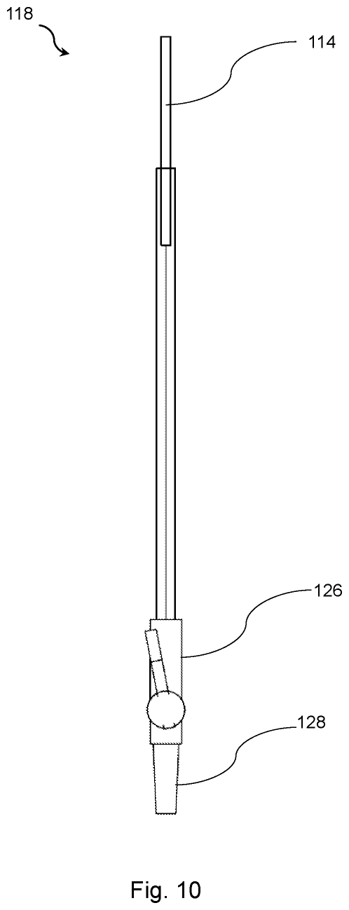

The urine collection device also includes a collection container 118. A second end 116 of the tube 112 is connected to the collection container 118. The collection container 118 can be used to collect the urine sample in use. The first end 115 of the tube 112 is sealed to the outlet 110 and the second end 116 of the tube 112 is sealed to the collection container 118. This prevents leakage of the urine sample from the urine collection device. Front and side views of the collection container 118 in isolation are shown in FIGS. 9 and 10, respectively. The collection container 118 includes an inlet valve (not shown) at an inlet end 117 of the collection container 118. In the urine collection device of the present embodiment, the inlet valve is a non-return valve. In embodiments, alternative inlet valves may be used. The collection container 118 also includes an outlet valve (not shown) at an outlet end 122 of the collection container 118. In the urine collection device of the present embodiment, the outlet valve is a non-return valve. In embodiments, alternative outlet valves may be used.

The urine collection device further comprises a stop tap 124 and nozzle 128 for controlling flow of the collected urine sample out of the collection container 118 in use. The stop tap 124 is sealed at the outlet end 122 of the collection container 118, which further prevents leakage of the urine sample from the urine collection device. The stop tap 124 may be attached to the collection container 118 by any suitable means, e.g., by injection moulding or heat-sealing. The nozzle 128 enables the urine to be emptied from the collection container 118 using the stop tap 124.

The collection container 118 shown in FIGS. 1, 2, 9 and 10 is in the form of a flexible pouch, which is lightweight, durable and versatile. The flexible pouch includes sealed edges to further prevent leakage of the urine sample from the urine collection device. The collection container 118 may have a capacity of, for example, 300 ml and may be made from a plastic material. The size and shape of the collection container 118 may vary depending on the intended use of the urine collection device. For example, the collection container 118 may have a height of approximately 15 cm and a width of approximately 12 cm for pre-continent users. The collection container 118 may have a capacity of approximately 500 ml for incontinent users. The collection container 118 may have a capacity of approximately 2 litres for incontinent users.

The collection container 118 includes an object support structure 114 for mounting the collection container 118 to a nearby object in use. The object support structure 114 is proximal to the inlet end 117 of the collection container 118. The object support structure 114 shown in FIGS. 1, 2, 9 and 10 is in the form of a flat-fold hook 114 which includes two U-shaped support structures. In an open configuration, the U-shaped support structures enable the collection container 118 to be mounted onto an object, such as a bed frame or to the back of a nappy, when the urine collection device is in use in order to keep the collection container 118 stable and/or out of sight. For incontinent users, the object support structure 114 can be used to position the collection container 118 away from the user's body, e.g., onto a bed frame or a stand. In a closed configuration, the U-shaped support structures can be folded into alignment such that the urine collection device can be stored more efficiently. For example, the U-shaped support structures can be folded such that they are substantially flush with the collection container 118. The object support structure 114 may be attached to the collection container 118 by any suitable means, e.g., by injection moulding or heat-sealing. The size and shape of the object support structure 114 may vary depending on the intended use of the urine collection device. For example, each of the U-shaped support structures may have a height of approximately 4 cm to 4.5 cm and a width of approximately 4 cm.

The collection container 118 also includes a user support structure 120, 121 for mounting the collection container 118 to an item of clothing of the user in use. The user support structure 120, 121 shown in FIGS. 1, 2, 9 and 10 includes a first support slot 120 at a first side of the collection container 118 and a second support slot 121 at a second side of the collection container 118. The first support slot 120 and the second support slot 121 are configured to receive a strap (not shown) for mounting the collection container 118 to the user in use. For example, the strap may be used to mount the collection container 118 to a leg or the waist of the user. The strap may be used to mount the collection container 118 to an item of clothing of the user. In embodiments, the dimensions and number of the support slots may vary in order to accommodate collection containers 118 of different shapes and sizes. The user support structure 120, 121 may be attached to the collection container 118 by any suitable means, e.g., by heat-sealing. In embodiments, the collection container 118 may be configured with different mounting options. The user support structure 120, 121 may be formed by any suitable means, e.g., by die-cutting.

The collection container 118 shown in FIGS. 1, 2, 9 and 10, which is in the form of a flexible pouch, is transparent and made of polyvinyl chloride (PVC). The transparency enables the amount of urine that has been collected to be viewed and monitored, and PVC provides a suitable material that is tough, durable and economical. The urine collection device is configured to be disposable. In embodiments, the urine collection device may be configured to be reusable.

The urine collection device may be classified as a Class 1 medical device, namely a device that has a low to moderate risk to the patient and/or user.

In use, the receptacle 100 is placed within a nappy or an incontinence pad of a user. The opening 102 of the receiving portion 101 is configured to engage with a human body portion surrounding external genitalia of the user. Urine exiting the body of the user is captured directly by the receptacle 100, without contacting the user's genitalia or skin during collection. The urine sample is received and retained by the reservoir 103. This prevents contact of the urine sample with the user's genitalia or skin after collection. The tube 112 acts as a conduit for the urine sample from the receptacle 100 to the collection container 118. Urine thereby flows from the reservoir 103 into the tube 112 via the outlet 110. The non-return inlet valve at the inlet end 117 of the collection container 118 prevents urine from being transported back into the tube 112 and back to the receptacle 100. Once the urine sample has been collected within the collection container 118, the urine sample can be removed from the collection container 118 easily and hygienically by opening the stop tap 124 at the outlet end 122 of the collection container 118. To remove the uncontaminated urine sample from the collection container 118, the stop tap 124 can be turned on to allow urine to flow through the nozzle 128. The stop tap 124 can be turned off to stop the flow of urine through the nozzle 128 when required. The urine collection device can therefore also be used to collect an uncontaminated urine sample without removing the receptacle 100 from the nappy or incontinence pad. This enables the uncontaminated urine sample to be collected in an efficient and discrete manner. As such, the user is not required to use a private bay or room, but instead can use the urine collection device in a public area, such as a hospital waiting room.

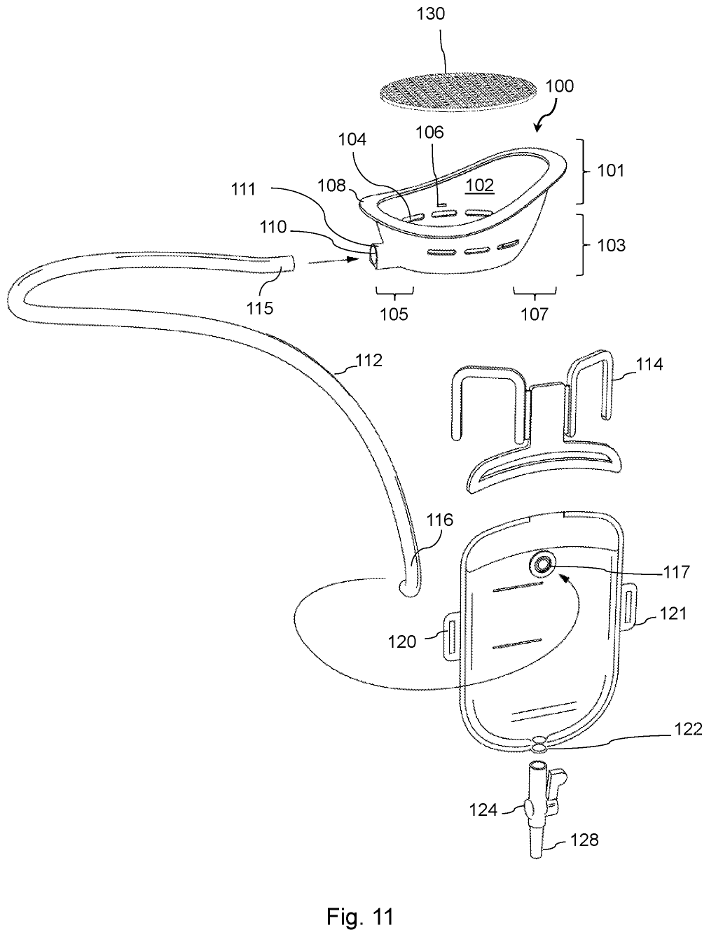

FIGS. 11, 12 and 13 show a urine collection device in accordance with a second embodiment of the present invention. The urine collection device shown in these figures includes the features of the urine collection device according to the first embodiment as described above with reference to FIGS. 1 to 8. The urine collection device according to the second embodiment also comprises a mesh guard 130. The mesh guard 130 may be removable. The mesh guard 130 is attached to the receptacle 100 at the receiving portion 101. The mesh guard 130 enables the urine collected in the reservoir 103 to be separated from the external genitalia of the user in use. The mesh guard 130 is a substantially flat component shaped to fit into the receptacle 100. The mesh guard 130 is formed of a network of material with a plurality of openings that allow urine to flow into the reservoir 103. The plurality of openings may be evenly-spaced. The plurality of openings may be uniform. In the mesh guard 130 shown in FIGS. 11, 12 and 13, the mesh size, i.e., the size of each opening, is 3 mm×3 mm, and the width of the network of material is 1 mm. Other mesh sizes may be used. For example, a mesh size between approximately 1 mm×1 mm and approximately 3 cm may be used, depending on the intended use of the urine collection device. Preferably, the mesh size is between approximately 1 mm×1 mm and 5 mm×5 mm. In embodiments, the mesh guard 130 may be formed of a network of material with non-uniform openings and/or openings that are not evenly spaced. The mesh guard 130 may be formed using injection moulding techniques.

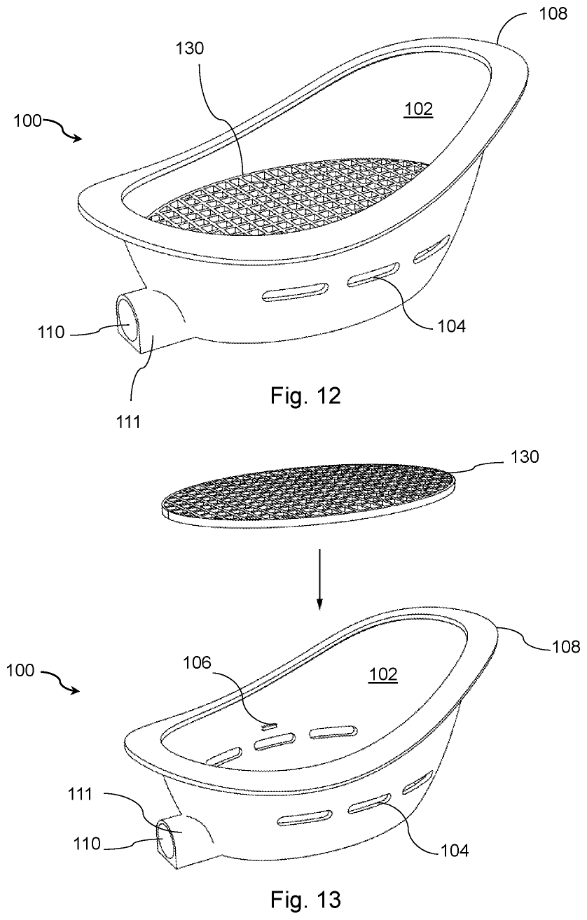

Using a mesh guard 130 as part of the urine collection device enables the urine collection device to be particularly suitable for collecting uncontaminated urine samples from larger pre-continent male users and incontinent male users, since the mesh guard 130 acts as a barrier to prevent male genitalia from entering the reservoir 103. FIG. 11 shows an exploded view of the urine collection device including the mesh guard 130, and FIGS. 12 and 13 depict how the mesh guard 130 can be attached to the receptacle 100. As can be seen in FIGS. 11 and 13, the receptacle 100 includes engagement clips 106 for attaching the mesh guard 130 to the receptacle 100. In order to attach the mesh guard 130 to the receptacle 100, the mesh guard 130 is positioned in alignment with the opening 102 of the receiving portion 101 of the receptacle 100 and then placed into the opening 102, as shown in FIG. 13. The mesh guard 130 is fitted into receptacle 100 by applying pressure in a downward motion until it is clipped into the engagement clips 106 disposed within the receptacle 100. The mesh guard 130 is held in place within the receptacle 100 using the engagement clips 106, as shown in FIG. 12. In embodiments, fewer or more engagement clips than shown may be used. The mesh guard 130 may comprise one or more support clips in order to more securely attach the mesh guard 130 to the receptacle 100. The one or more support clips may be configured to engage with the engagement clips 106 disposed within the receptacle 100. In embodiments, the mesh guard 130 may be attached to the receptacle 100 using a press fit or friction fit fitting.

As shown in FIG. 12, the overflow slots 104 are positioned between the mesh guard 130 and the reservoir 103. Advantageously, this enables excess urine to be released via the overflow slots 104 before the level of the urine sample reaches the mesh guard 130, which further prevents contact of the urine sample with the user's genitalia or skin.

The mesh guard 130 may be made from any suitable material which enables it to prevent male genitalia from entering the reservoir 103 and can be fitted within the receptacle 100. The mesh guard 130 may comprise a degree of flexibility to provide comfort for the user. For example, the mesh guard 130 may be made from a material which is rigid enough to prevent male genitalia from entering the reservoir 103 and to prevent it from bending out of shape when being fitted into the receptacle 100, yet flexible enough to be comfortable for the user when the urine collection device is in use.

The receptacle 100, the mesh guard 130 and/or the tube 112 comprises a flexible material to provide further comfort for the user. For example, the receptacle 100, the mesh guard 130 and/or the tube 112 may be made of medical-grade silicone, which is otherwise known as liquid silicone rubber, for external use. Medical-grade silicone satisfies the International Organization for Standardization (ISO) standard for medical grade materials, namely ISO 10993, and is skin-contact safe, durable, flexible, and soft to the touch. In embodiments, the receptacle, the mesh guard and/or the tube may be made of an alternative skin-contact safe material. In embodiments, the receptacle, the mesh guard and/or the tube may be made of a polymer material.

The mesh guard 130 may contour the entire inner wall of the receptacle 100, or the mesh guard 130 may partially contour the inner wall of the receptacle 100 such that part of the opening 102 is covered by the mesh guard 130.

Many modifications may be made to the specific embodiments described above without departing from the scope of the invention as defined in the accompanying claims. Features of one embodiment may also be used in other embodiments, either as an addition to such embodiment or as a replacement thereof.

What is claimed is:

1. A urine collection device for collecting an uncontaminated urine sample from a pre-continent or an incontinent user, the urine collection device comprising:
a receptacle configured to be placed within a nappy or an incontinence pad of the user to receive and retain a urine sample collected from the user in use, wherein the receptacle comprises:
a receiving portion for receiving the urine sample, the receiving portion comprising an opening configured to engage with a human body portion surrounding external genitalia of the user, thereby enabling urine exiting the body of the user to be captured directly in use;
a reservoir for retaining the received urine sample in use;
an outlet for draining the retained urine sample in use, the opening being larger than the outlet; and
wherein the receptacle has a greater height dimension at a forward-facing end than a rearward-facing end, and wherein the outlet is positioned at the rearward-facing end.

2. The urine collection device of claim 1, wherein the receptacle is between approximately 10 cm and approximately 17 cm in length, between approximately 4 cm and approximately 10 cm in width, and between approximately 3 cm and approximately 8 cm in height.

3. The urine collection device of claim 1, wherein the reservoir comprises a substantially flat base.

4. The urine collection device of claim 3, wherein the outlet is in alignment with the substantially flat base.

5. The urine collection device of claim 1, wherein the receptacle comprises at least one overflow opening for releasing excess urine in use.

6. The urine collection device of claim 1, further comprising a mesh guard at the receiving portion for separating the urine collected in the reservoir from the external genitalia of the user in use.

7. The urine collection device of claim 5, further comprising a mesh guard at the receiving portion for separating the urine collected in the reservoir from the external genitalia of the user in use and wherein the at least one overflow opening is located between the mesh guard and the reservoir.

8. The urine collection device of claim 6, wherein the mesh guard is attached to the receptacle using a press fit fitting.

9. The urine collection device of claim 6, wherein the receptacle comprises at least one engagement clip and the mesh guard comprises at least one support clip for attaching the mesh guard to the receptacle.

10. The urine collection device of claim 1, wherein the urine collection device further comprises a tube, a first end of the tube being connected to the outlet, for directing the retained urine sample away from the reservoir in use.

11. The urine collection device of claim 10, further comprising a mesh guard at the receiving portion for separating the urine collected in the reservoir from the external genitalia of the user in use, wherein the receptacle, the mesh guard and/or the tube comprises a flexible material.

12. The urine collection device of claim 11, wherein the flexible material of the receptacle has a variable degree of flexibility characteristic and the degree of flexibility of the receptacle at the receiving portion is greater than the degree of flexibility of the receptacle at the reservoir.

13. The urine collection device of claim 10, further comprising a mesh guard at the receiving portion for separating the urine collected in the reservoir from the external genitalia of the user in use, wherein the receptacle, the mesh guard and/or the tube comprises medical-grade silicone.

14. The urine collection device of claim 10, further comprising a collection container, a second end of the tube being connected to the collection container, for collecting the urine sample in use and the first end of the tube is sealed to the outlet and the second end of the tube is sealed to the collection container.

15. The urine collection device of claim 14, wherein the collection container comprises an non-return inlet valve at an inlet end of the collection container.

16. The urine collection device of claim 14, wherein the collection container further comprises a non-return outlet valve at an outlet end of the collection container.

17. The urine collection device of claim 14, further comprising a stop tap for controlling flow of the collected urine sample out of the collection container in use.

18. The urine collection device of claim 14, wherein the collection container comprises an object support structure for mounting the collection container to a nearby object in use.

19. The urine collection device of claim 18, wherein the object support structure comprises a flat-fold hook.

20. The urine collection device of claim 14, wherein the collection container comprises a user support structure for mounting the collection container to an item of clothing of user in use.
